(19) United States
(12) Patent Application Publication
(10) Pub. No.: US 2008/0267865 A1
(43) Pub. Date: Oct. 30, 2008

(54) Targeting of Erb Antigens

(75) Inventors: Bengt E.B. Sandberg, Hjarup (SE); Rune Nilsson, Lund (SE)

Correspondence Address: SWANSON & BRATSCHUN, L.L.C., 8210 SOUTHPARK TERRACE, LITTLETON, CO, 80120, US

(73) Assignee: Biotech IgG AB

(21) Appl. No.: 10/596,012

(22) PCT Filed: Nov. 26, 2004
(86) PCT No.: PCT/SE04/01753

§ 371 Date: Jun. 25, 2008

Related U.S. Application Data
(60) Provisional application No. 60/525,703, filed on Nov. 28, 2003.

(30) Foreign Application Priority Data
Nov. 28, 2003 (SE) 0303229-9

(51) Int. Cl. A61K 51/10; A61K 39/395

(52) U.S. Cl. 424/1.53; 424/179.1; 424/9.1

(57) ABSTRACT

A conjugate comprising
a) a trifunctional cross-linking moiety, to which is coupled
b) an affinity ligand via a linker 1,
c) a cytotoxic agent, optionally via a linker 2, and
d) an anti Erb antibody or variants thereof having the ability to bind to Erb antigens expressed on mammalian tumour surfaces with an affinity-binding constant of at least 5×10⁶ M⁻¹,
wherein the affinity ligand is biotin, or a biotin derivative having essentially the same binding function to avidin or streptavidin as biotin, wherein stability towards enzymatic cleavage of the biotinamide bond has been introduced in linker 1.

### TECHNICAL FIELD OF THE INVENTION

The present invention relates to a conjugate and a novel medical composition comprising said conjugate which binds to mammalian Erb gene products, to a kit comprising the medical composition and an extracorporeal device, and to methods for treatment and/or diagnosing of cancer expressing Erb gene products.

### BACKGROUND ART

Proto-oncogenes that encode growth factors and their receptors contribute to the development of breast cancer and other human malignancies (Aronson, S A, Science, 254: 1146-1153 (1991) and, therefore, are potential targets for novel therapeutic strategies. In particular, increased expression of this gene has been observed in more aggressive carcinomas of the breast, bladder, lung and stomach.

The human epidermal growth factor receptor-2 (HER2) encodes a cell-surface receptor and is involved in signal transduction pathways that are responsible for normal cell growth and differentiation (DiAgustine R & Richards R G, J. Mammary Gland Biol Neoplasia 2:109-118 (1997); . However, the HER2 receptor is overexpressed in 15 to 25% of human breast cancers (Hynes N E & Stern D F, 1198:165-184 (1994), Revillion F et. al., Eur. J. Cancer 34:791-808 (1998) and such overexpression is correlated with poor clinical outcome in women with node-positive and node-negative disease, including reduced disease-free and overall survival (Hynes N E & Stern D F, Biochim. Biophys. Acta, 1198:165-184 (1994); Slamon D J et. al. Science, 244:707-712; Ravdin P M & Chamness G C, Gene, 159:19-27 (1995); Bell R. Oncology, 63(supp1.1): 39-46 (2002). Further, current evidence suggests that HER2 is predictive for response to standard anticancer therapies. See also PCT/US00/18283; PCT/US97/18385; PCT/US98/26266; EP 1 106 183; PCT/US00/12552 and PCT/US00/17366.

HER-2 is a member of the erbB epidermal growth factor receptor tyrosine kinase family. In the early 1980s the erbB receptor tyrosine kinases became implicated in cancer when it was found that the avian erythroblastosis tumor virus encoded an oncogene that was highly homologous to the human epidermal growth factor receptor (HER-1, also known as ErbB1 and EGFR). Subsequently a gene called neu was identified from a chemically induced rat neuroblastoma that was able to transform fibroblast cell lines in culture and was shown to be related to but distinct from the HER-1 gene (Shih, C et al., Nature, 290:261-264 (1981), Schechter et al., Nature, 312:513-516 (1984). At about the same time two other groups independently isolated human erbB-related proto-oncogenes and named them HER-2 (Coussens et al., Science, 230: 1132-1139 (1985) and c-erbB2 (Semba et al., PNAS, 82: 6497-6501 (1985). These genes were then shown to be the same as neu. King and colleagues also identified an EGFR-related gene that was over-amplified in a human mammary carcinoma cell line; this gene was also found to be identical to the HER-2/neu/erbB2 gene (King, C R. et al., Science 229:974-976 (1985).

HER-1 and HER-2 differ in a number of ways: the HER-2 gene is located on chromosome 17 whereas the HER-1 gene has been mapped to chromosome 7, and the HER-2 mRNA and protein are of different sizes from the HER-1 gene products. The erbB receptor tyrosine kinase family has two other members, HER-3 and HER-4 (erbB4), with the four receptors sharing an overall membrane spanning structure composed of extracellular and transmembrane components together with an intracellular region containing a kinase domain flanked by tyrosine autophosphorylation sites.

There are a number of functional differences between the domains of the different family members. For example, HER-2 appears to have no direct ligand and HER-3 has no intrinsic kinase activity and therefore a number of complex interactions between the different family members involving dimerisation are required for signalling. The HER-2 receptor can signal by forming heterodimers with other members of the HER family that are bound to a ligand, or two HER-2 molecules can combine to form a homodimer which has intrinsic kinase activity. Overexpression of HER-2 favours the production of both activated recruits of homo- and hetero-dimers. ErbB receptor kinase activation recruits a number of adaptor proteins to the cytoplasmic domains which in turn trigger a number of downstream signalling cascades. The end results of HER-2 activation are effects on cell growth, division, differentiation, migration and adhesion/reviewed in Yarden, Y & Sliwkowski, M X, Nature Reviews in Molecular and Cellular Biology, 2: 127-137 (2001).

Slamon and colleagues initially reported that the HER-2 receptor was overexpressed in 20-30% of human breast cancers (Slamon, D J et al., Science 235:177-182 (1987). In the vast majority of cases overexpression is caused by amplification of the HER-2 gene (Pauletti, G et al., Oncogene, 13:63-72 (1996). Amplification and/or overexpression of the human HER2 gene correlates with a poor prognosis in breast and ovarian cancers (Slamon, D J et al., Science, 235:177-182 (1987); and Slamon, D J et al., Science, 244:707-712 (1989)). Overexpression of HER2 has also been correlated with other carcinomas including carcinomas of the stomach, endometrium, salivary gland, lung, kidney, colon and bladder. HER-2 gene amplification results in increased levels of mRNA as detected by Northern blot and of the HER-2 receptor as detected by immunohistochemistry (IHC) or Western blot analysis. Over-amplification of the gene is most strikingly seen using fluorescence in situ hybridisation (FISH), when multiple copies of the HER-2 gene can be seen in the nuclei of affected cells. This technique has become a useful method of detecting HER-2 gene amplification in clinical samples.

A further related gene, called erbB3 or HER3, has also been described. See U.S. Pat. Nos. 5,183,884 and 5,480,968; Plowman et al., Proc. Natl. Acad. Sci. USA, 87:4905-4909 (1990); Kraus et al., Proc. Natl. Acad. Sci. USA, 86:9193-9197 (1989); EP Pat Appln No 444,961a1; and Kraus et al., Proc. Natl. Acad. Sci. USA, 90:2900-2904 (1993). Kraus et al. (1989) discovered that markedly elevated levels of erbB3 mRNA were present in certain human mammary tumor cell lines indicating that erbB3, like erbB1 and erbB2, may play a role in some human malignancies. These researches demonstrated that some human mammary tumor cell lines display significant elevation of steady-state ErbB3 tyrosine phosphorylation, further indicating that this receptor may play a role in human malignancies. Accordingly, diagnostic bioassays utilizing antibodies, which bind to ErbB3, are described by Kraus et al. in U.S. Pat. Nos. 5,183,884 and 5,480,968.

The role of erbB3 in cancer has also been explored by others. It has been found to be overexpressed in breast (Lemoine et al., Br. J. Cancer, 66:1116-1121 (1992)), gastrointestinal (Poller et al., J. Pathol., 168:275-280 (1992), Rajkumer et al., J. Pathol., 170:271-278 (1993), and Sanidas et al., Int. J. Cancer. 54:935-940 (1993)), and pancreatic cancers (Lemoine et al., J. Pathol., 168:269-273 (1992) and Friess et al. Clinical Cancer Research, 1:1413-1420 (1995)).

ErbB3 is unique among the ErbB receptor family in that it possesses little or no intrinsic tyrosine kinase activity (Guy et al., Proc. Natl. Acad. Sci. USA 91:8132-8136 (1994) and Kim et al. J. Biol. Chem. 269:24747-55 (1994)). When Erb3 is co-expressed with ErbB2 an active signaling complex is formed and antibodies directed against ErbB2 are capable of disrupting this complex (Sliwkowski et al., J. Biol. Chem., 269(20): 14661-14665 (1994)). Additionally, the affinity of ErbB3 for heregulin (HRG) is increased to a higher affinity state when co-expressed with ErbB2. See also Levi et al., Journal of Neuroscience 15: 1329-1340 (1995): Morrissey et al., Proc. Natl. Acad. Sci. USA 92:1431-1435 (1995); and Lewis et al., Cancer Res., 56:1457-1465 (1996) with respect to the ErbB2-ErbB3 protein complex.

Rajkumar et al., British Journal Cancer. 70(3):459-465 (1994). Developed a monoclonal antibody against ErbB3, which had an agonistic effect on the anchorage-independent growth of cell lines expressing this receptor.

The class 1 subfamily of growth factor receptor protein tyrosine kinases has been further extended to include the HER4/p180erbB4 receptor (See EP Pat Appln No 599,274; Plowman, et al., Proc. Natl. Acad. Sci. USA, 90:1746-1750 (1993); and Plowman et al., Nature, 366:473-475 (1993). Plowman et al. found that increased HER4 expression closely correlated with certain carcinomas of epithelial origin, including breast adenocarcinomas. Accordingly, diagnostic methods for detection of human neoplastic conditions (especially breast cancers) which evaluate HER 4 expression are described in EP Pat Appln No. 599,274.

The search for an activator of the HER2 oncogene has lead to the discovery of a family of heregulin polypeptides. These proteins appear to result from alternative splicing of a single gene which was mapped to the short arm of human chromosome 8 by Lee et al., Genomics, 16:790-791 (1993); and Orr-Urtreger et al., Proc. Natl. Acad. Sci. USA, Vol. 90 pp. 1867-1871 (1993); PCT/US79/03546 and PCT/US97/11825.

The discovery of HER-2 overexpression in a significant minority of human breast cancers and its adverse prognostic significance prompted investigators to develop agents using HER-2 as a target for treatments. Several groups including workers at Genentech Inc. raised murine monoclonal antibodies to the extra cellular domain of HER-2 and showed that some of these antibodies were capable of inhibiting the growth of cell lines that overexpressed the receptor (Hudziak, R M, et al Molecular Cell Biology, 9:1165-1172 (1989); Fendly, B M., et al. Cancer Research 50:1550-1558 (1990). This effect was also seen in HER-2-overexpressing human breast cancer xenografts where the effects of the antibody were found to be synergistic to anti-neoplastic agents such as cisplatin (Pietras, R J et al., Cancer Research, 9: 1829-1838 1994); Harris, M & Smith, I, Endocrine-Related Cancer, 9: 75-85 (2002).

The Genentech researchers developed a panel of murine monoclonal antibodies capable of inhibiting HER-2+ cell lines; the most potent of these was muMAb 4D5. This antibody was found markedly to inhibit proliferation of cell lines that overexpressed HER-2 but had little or no effect on cells without elevated levels of HER-2 (Sarup, J C. et al., Growth Regulation, 1: 72-82 (1991). 4D5 was found to be a potent inhibitor of growth of human breast cancer xenografts (Beselga & Mendelsohn, Pharmacology Therapy, 64: 127-154 (1994) and was therefore selected for further clinical development.

In order to reduce the potential for generating a human anti-mouse immune response the 4D5 murine monoclonal antibody was subsequently humanised. Carter and colleagues subcloned the hypervariable region of the antibody into plasmids encoding a human K light chain and the IgG1 constant region to generate a vector encoding a chimeric antibody which was then further humanised by site-directed mutagenesis (Carter, P., et al., PNAS: 89, 4285-4289 (1992). The vector was transduced into Chinese hamster ovary (CHO) cells that then secrete the antibody into the culture medium from which it is purified. The chimeric antibody called trastuzumab is 95% human and 5% murine and retains the high affinity for the HER-2 epitope of the parental antibody.

Trastuzumab has a binding affinity for HER-2 that is three times that of its parent murine antibody 4D5. Like 4D5, it has been shown to have a marked anti-proliferative effect on HER-2-overexpressing cell lines and very little effect on cells not expressing HER-2 (Carter, P. et al., PNAS: 89, 4285-4289 (1992). This anti-proliferative effect has also been demonstrated in vivo in breast cancer xenograft experiments by Baselga and colleagues in which established BT-474 tumour xenografts were inhibited from growing by trastuzumab. In doses of less than 1 mg/kg growth was inhibited in a dose-dependent fashion and no growth at all was seen at higher doses (Baselga, J. et al., Cancer Research, 58: 2825-2831 (1998). In the same study, the researchers explored the addition of trastuzumab to either paclitaxel or doxorubicin. Chemotherapy alone was shown to have only modest anti-tumor activity, whereas combined treatment with trastuzumab resulted in a marked enhancement of the effect of chemotherapy with the greatest growth inhibition being seen with paclitaxel and trastuzumab.

Pegram and colleagues examined the effect of trastuzumab on a number of other chemotherapeutic agents in a HER-2 transfected MCF7 xenograft model. Synergistic interactions were seen with cisplatin, docetaxel, thiotepa, cyclophosphamide, vinorelbine and etoposide. Addictive effects were seen with doxorubicine, paclitaxel, vinblastine and methotrexate and the combination of trastuzumab with 5-fluorouracil (5-FU) was found to be antagonistic (Pegram, M. et al., Oncogene, 18: 2241-2251 (1999); Konecny, G, et al., Breast Cancer Research and Treatment, 69:53-63 (2001) and reviewed in Pegram, M D. et al., Seminars in Oncology, 27: 21-25 (2000). The synergy seen in these in vivo models has led to the exploration in clinical trials of trastuzumab in combination with chemotherapy.

Trastuzumab (Herceptin®) has been shown to provide significant clinical benefits in patients with HER2-positive metastatic breast disease when administered as monotherapy (Cobleigh M A et al. J. Clin. Oncol. 17:2639-2648 (1999); Vogel C L. Et. al. J. Clin. Oncol. 20:719-726 (2002) or in combination with chemotherapy Slamon D J. et. al. N. Engl. J. Med. 344:783-792 (2001). Trastuzumab therapy is associated with impressive survival benefits (Vogel C L. et. al. J. Clin. Oncol. 20:719-726 (2002); Slamon D J. et. al. N. Engl. J. Med. 344:783-792 (2001), including a 45% increase in median survival when it is added to chemotherapy (29 vs 20 months, respectively) in patients whose tumours demonstrate IHC 3+ protein overexpression by immunohistochemistry (IHC) compared with chemotherapy alone (Smith I E, Anticancer Drugs 12 (suppl. 4): S3-S10 (2001). As indicated elsewhere in this supplement, evidence from cross-trial comparisons suggests that, in the metastatic setting, the clinical benefits achieved with trastuzumab are greater the earlier treatment is given (Bell R. Oncology, 63 (suppl.1): 39-46 (2002).

WO 03/03511 (The AB Research Foundation) discloses multidrug multiligand conjugates for targeted drug delivery, wherein an epidermal growth factor receptor recognizing peptide, a monoclonal antibody or a portion thereof may be used as targeting molecules.

WO 01/00244 (Genentech, Inc.) discloses methods of treatment using anti-ErbB antibody-maytansinoid conjugates, wherein the maytansinoid is directly bound to the anti-ErbB antibody.

WO 00/02050 (Mitra Medical Technology AB and Department of Radiation Oncology, University of Washington) discloses a trifunctional reagent for conjugation to a biomolecule.

An estimated 211,300 new cases of invasive breast cancer are expected to occur among women in the United States during 2003. It is the most frequently diagnosed non-skin cancer in women. Breast cancer incidence rates have continued to increase since 1980, although the rate of increase slowed in the 1990s, compared to the 1980s. Furthermore, in the more recent time period, breast cancer incidence rates have increased only in those aged 50 and over. About 1,300 new cases of breast cancer are expected in men in 2003.

In addition to invasive breast cancer, 55,700 new cases of in situ breast cancer are expected to occur among women during 2003. Of these, approximately 85% will be ductal carcinoma in situ (DCIS). The increase in detection of DCIS cases is a direct result of increased use of screening with mammography, with detects invasive breast cancers before they are palpable, that is, before they can be felt.

An estimated 40,200 deaths (39,800 women, 400 men) anticipated from breast cancer ranks second among cancer deaths in women. According to the most recent data, mortality rates declined by 1.4% per year during 1989-1995 and by 3.2% afterwards, with the largest decrease in younger woman in both whites and African Americans. These decreases are probably the result of both earlier detection and improved treatment.

Despite the fact that tumors are removed by surgery, there is always a risk of recurrence because there may be microscopic cancer cells that have spread to distant sites in the body. In order to decrease a patient's risk of recurrence, many breast cancer patients are offered chemotherapy. Chemotherapy is the use of anti-cancer drugs that go throughout the entire body.

There are many different chemotherapy drugs, and they are usually given in combinations for 3 to 6 months after the patient received her surgery. Depending on the type of chemotherapy regimen received, medication may be given every 3 or 4 weeks and many of the drugs have to be given systemically. Two of the most common regimens are AC (doxorubicin and cyclolphophamide) for 3 months or CMF (cyclophosphamide, methotrexate, and fluorouracil) for 6 months.

Sometimes patients have a recurrence of their cancer, or progress to stage IV with disease outside their breast. These patients will all need chemotherapy, and a variety of different agents may be tried until a response is achieved. Sometimes chemotherapy is given before surgery, i.e. neoadjuvant chemotherapy. This is usually reserved for very advanced cancers that need to be shrunken before they can be operated on.

Breast cancer commonly receives high energy radiation-therapy, which requires patients to come 5 days a week for up to 6 weeks to a radiation therapy treatment center. Radiation is important in reducing the risk of local recurrence and is often offered in more advanced cases to kill tumor cells that may be located in lymph nodes.

Although, trastuzumab (Herceptin) has shown to increase the “mean survival time” for breast cancer in patients over-expressing Her-2, the most significant effect occurs when combined with chemotherapy. However, these combined therapies are afflicted with severe side effects, in particular ventricular dysfunction and congestive heart failure, which has in some cases been fatal. The incidence and severity of cardiac dysfunction was particularly high in patients who received Herceptin in combination with anthracyclines and cyclophosphamid.

Radioimmunotargeting has proven to be more effective than the naked antibody for a number of cancer indications (Goldenberg D. M. & Nabi, H. A., Cancer 89:104-113, 2000).

Whereas the efficacy of “naked antibodies” relies on the ability to induce host tumour response via antibody-dependent cell toxicity (ADCC) and complement activation or as in the case of trastuzumab (Herceptin) block and possibly prevent further growth by interrupting the growth signal. Radiolabelled antibodies, on the other hand, kill tumour cells by emission of radioactive particles and may therefore be effective even when host immune-effector functions are impaired. Furthermore, dependent on radionuclide characteristics, radioimmunotherapy is capable of destroying cells distant from immunotargeted cells (cross firing). Consequently, even heterogeneous tumours (tumours that express various degrees of the antigen) can be treated, because not all cells have to be targeted. Hence, antibodies carrying radio nuclides only require tumour specific binding sites in order to exert their cell-killing effect. However, radioimmunotargeting may also be used in conjunction with the naked antibody and/or together with chemotherapy or external irradiation.

Several studies have explored the use of radioimmunotargeting in breast cancer. Antigen targets have included primarily CEA, MUC1, and L6. These and other antibodies used in breast cancer have recently been reviewed (Goldenberg D. M. & Nabi, H. A., Cancer 89:104-113, 2000).

However, normal organ toxicity limits the amount of activity that can safely be administered to patients and thereby the absorbed dose to tumour. The first dose-limiting organ is the bone marrow. Hemotological cancer like localised B-cell lymphoma may be cured by external beam radiotherapy with a dose of 30 to 44 Gy. The dose that may be achieved with conventional radioimmunotherapy without the use of stem cell support is substantially lower. Wiseman et al has reported a median dose of 15 Gy in B-cell lymphoma in a phase III trial (Wiseman G et al., Critical reviews in Oncology/Hematology 39 (2001) 181-194). The response rate was 80% objective response and 34% complete response. The Seattle group using stem cell support has reported the highest remission rate 80% complete remissions (Liu Steven Y. et al., J. Clin. Oncol. 16(10): 3270-3278, 1998). They estimated tumour sites to achieve 27 to 92 Gy.

The non-haematological dose-limiting toxicity was reversible pulmonary insufficiency, which occurred at doses ≧27 Gy to the lungs. Although the studies are not quite comparable, they indicate a dose effect relationship in RIT. If there is a dose relationship, it may be possible to increase efficacy if a higher dose to the tumour can be delivered. This may be most clinically relevant, since complete remission following RIT has been associated with longer duration of remission (Wahl et al., J. Nucl. Med. 39:21 S-26S, 1998.).

An obstacle to this is the radio sensitivity of the bone marrow. A higher absorbed dose to the bone marrow may cause myeloablation. Thus, the dose necessary to achieve a more effective therapy is hampered by the accumulation of radioactivity in the blood circulation, leading to toxicity of normal organs, such as bone marrow. Various means to clear blood from cytotoxic targeting biomolecules (e.g. therapeutic or diagnostic monoclonal antibodies) after intravenous administration have been reported (See review article by Schriber G. J. and Kerr D. E., Current Medical Chemistry 2:616-629, (1995); Goldenberg D. M., J. Nucl. Med 43: 693-713 (2002) and Carlsson et. al. Radiotherapy and oncology 66: 107-117 (2003).

Various methods have been proposed to rapidly clear radiolabelled antibodies from blood circulation after the tumour has accumulated a sufficient quantity of immunoconjugate to obtain a diagnosis or therapy. Some of the methods employed involve enhancement of the body's own clearing mechanism through the formation of immune complexes. Enhanced blood clearance of radiolabelled antibodies can be obtained by using molecules that bind to the therapeutic antibody, such as other monoclonal antibodies directed towards the therapeutic antibody (Klibanov et al, J. Nucl. Med. 29:1951-1956 (1988); Marshall et al, Br. J. Cancer 69: 502-507 (1994); Sharkey et al, Bioconjugate Chem. 8:595-604, (1997), avidin/streptavidin (Sinitsyn et al J. Nucl. Med. 30:66-69 (1989), Marshall et al Br. J. Cancer 71:18-24 (1995), or glycosyl containing compounds which are removed by receptors on liver cells (Ashwell and Morell Adv. Enzymol. 41:99-128 (1974).

In the so-called avidin chase modality, avidin or streptavidin is administered systemically after administration of the therapeutic or diagnostic antibody to which biotin has been attached, at a time when a sufficient amount of the antibody has been accumulated in the tumour. Avidin or streptavidin will associate with the antibodies and the so formed immunocomplex will be cleared from the blood circulation via the reticuloendothelial system (RES) and be cleared from the patient via the liver. These procedures will improve the clearance of biotinylated cytotoxic antibodies. An alternative approach to the same end is the use of anti-idiotypic antibodies. However, all these methods rely on the liver or kidney for blood clearance and thereby expose either or both of these vital organs as well as the urinary bladder to high dose of cytotoxicity.

Another major drawback of the methods is the immunogenicity of these agents, particularly the streptavidin, which prevent repetitive treatments once the immune response has been developed.

Extracorporeal techniques for blood clearance are widely used in kidney dialysis, where toxic materials build up in the blood due to the lack of kidney function. Other medical applications, in which an extracorporeal apparatus can be used, include: removal of radioactive materials; removal of toxic levels of metals, removal of toxins produced from bacteria or viruses; removal of toxic levels of drugs, and removal of whole cells (e.g cancerous cells, specific haematopoietic cells—e.g. B, T, or NK cells) or removal of bacteria and viruses.

The extracorporeal techniques used to clear a medical agent from blood circulation are particularly attractive because the toxic material is rapidly removed from the body.

Applications of these methods in the context of immunotargeting have been previously described (Henry Chemical Abstract 18:565 (1991); Hofheinze D. et al Proc. Am. Assoc. Cancer Res. 28:391 (1987); Lear J. K. et al Antibody Immunoconj. Radiopharm. 4:509 (1991); Dienhart D. G. et al Antibody Immunoconj. Radiopharm. 7:225 (1991); DeNardo S. J. et al J. Nucl. Med. 33:862-863 (1992); DeNardo G. L. et al J. Nucl. Med. 34:1020-1027 (1993); DeNardo G. L. J. Nucl. Med. 33:863-864 (1992); and U.S. Pat. No. 5,474,772 (Method of treatment with medical agents).

To make the blood clearance more effective and to enable processing of whole blood, rather than blood plasma as the above methods refer to, the medical agents (e.g. tumour specific monoclonal antibody carrying cell killing agents or radio nuclides for tumour localization) have been biotinylated and cleared by an avidin-based adsorbent on a column matrix. A number of publications provide data showing that this technique is both efficient and practical for the clearance of biotinylated and radionuclide labelled tumour specific antibodies (Norrgren K. et al, Antibody Immunoconj. Radiopharm. 4:54 (1991), Norrgren K. et al J. Nucl. Med. 34:448-454 (1993); Garkavij M. et al Acta Oncologica 53:309-312 (1996); Garkavij M. et al, J. Nucl. Med. 38:895-901 (1997)).

These techniques are also described in EP 0 567 514 and U.S. Pat. No. 6,251,394. The device MitraDep®, developed and manufactured by Mitra Medical Technology AB, Lund, Sweden, is based on this technology. By using the avidin-coated filter in conjunction with biotin labelled therapeutic antibodies, the blood clearance technique can be applied equally well for chimeric or fully humanised antibodies. Experimental data reveal that during a three-hour adsorption procedure, more than 90 percent of the circulating biotinylated antibodies can be removed by the MitraDep® system (Clinical Investigator's Brochure—MitraDep®). This has been confirmed in recent clinical studies.

In order to be adsorbed to the extracorporeal filter, the monoclonal antibodies carrying the cytotoxic agent (e.g. radionuclide) need to be biotinylated (biotin binds irreversibly to the avidin in the filter) prior to administration to the patient. The number of biotinyl moieties per IgG molecule is in the range of 3-6, typically 4.

However, in most cases the same type of functions (ε-amino groups) on the antibodies is utilized for coupling of the chelating groups and the biotinyl groups, leading to a competition of the most accessible sites.

Chelation and/or biotinylation of an antibody results in a heterogenous preparation, if for example a chelated antibody has an average of 3 chelates per antibody, the preparation will in fact contain a mixture of antibodies which range from 1 chelate/antibody to 7 chelates/antibody. As the chelate and biotin are linked to the same moieties on the antibody, some antibodies with a higher number of chelates will also have a low number of biotin molecules and some antibodies with a high number of chelates will have no biotin at all.

This means, statistically, that a population of the antibodies carrying radionuclide but no biotin will circulate in the blood, and these antibodies will not be removed by the MitraDep® filter.

To facilitate the labelling of the naked therapeutic or diagnostic antibody and to ensure that the ratio of biotin to the radiolabel is one to one, Mitra Medical Technology AB, Lund, Sweden has developed a series of novel water soluble structures (Tag-reagent; MitraTag™) containing the two types of functions, thereby enabling simultaneous and site specific conjugation of chelating groups (for radiolabelling) and the biotin groups.

This later method has a number of advantages over the consecutive labelling of radio nuclides and biotinylation and is particularly attractive in cases where the naked (non-chelated) antibody is supplied to the hospital, and where both the chelating group and the biotin groups have to be conjugated to the antibody in addition to the radiolabelling step.

A further development and applications of these agents are described in U.S. Pat. No. 6,251,394; PCT/SE98/01345; PCT/SE99/01241; PCT/SE99/01241; U.S. Ser. No. 09/519,998; U.S. Ser. No. 09/750,280; PCT/SE02/01191 and by Wilbur, S. D, et. al. Bioconjugate Chemistry, 13: 1079-1092 (2002).

The Tag-reagent labeled with the chelating group DOTA, is called MitraTag™-1033, as also stated in the definition part below.

### SUMMARY OF THE INVENTION

The object of the present invention is to solve the above discussed problems in connection with treatment of certain cancer diseases expressing the protooncogen Erb. This object is achieved by the present invention as defined in the claims and in the description below.

The present invention encompasses a conjugate including an anti Erb antibody, a medical composition comprising the conjugate including the anti Erb antibody, a kit comprising the medical composition, and various methods for the treatment and/or diagnosing of cancer expressing the oncogene protein HER, i.e. breast cancer and ovarian cancer in particular.

More precisely, the present invention relates in one aspect to a conjugate comprising
a) a trifunctional cross-linking moiety, to which is coupled
b) an affinity ligand via a linker 1,
c) a cytotoxic agent, optionally via a linker 2, and
d) an anti Erb antibody or variants thereof having the ability to bind to Erb antigens expressed on mammalian tumour surfaces with an affinity-binding constant of at least 5×10⁶M⁻¹, wherein the affinity ligand is biotin, or a biotin derivative having essentially the same binding function to avidin or streptavidin as biotin, wherein stability towards enzymatic cleavage of the biotinamide bond has been introduced in linker 1.

In another aspect the present invention relates to a medical composition comprising said conjugate and a pharmaceutically acceptable excipient.

In a further aspect the present invention relates to a kit for extracorporeal removal of, or at least reduction of, the concentration of the non-tissue bound medical composition comprising the conjugate in the plasma or whole blood of a mammalian host, wherein said medical composition previously has been introduced in the body of said mammalian host and kept therein a certain time in order to be concentrated to the specific tissues or cells by being attached thereto, said kit comprising

a) said medical composition, and

b) an extracorporeal device comprising an immobilized receptor onto which the affinity ligand of the reagent adheres.

In a further aspect, the present invention relates to Methods according to claims 33-45 for treatment and/or diagnosing of cancer expressing Erb gene products on the surface of its tumour cells in a mammalian host, wherein the medical composition is administered to the mammal in need thereof.

Further advantages and objects of the present invention will now be described in more detail, inter alia with reference to the accompanying drawings.

### BRIEF DESCRIPTION OF THE DRAWINGS

FIG. 1 shows competitive inhibition of ¹¹¹In labelled 1033-trastuzumab binding to SKBR-3 cells by cold (unlabelled, without 1033-conjugate) trastuzumab.

FIG. 2 shows comparison of whole body clearance of radioactivity in rats, injected with ¹¹¹In-1033-trastuzumab (filled triangles) or ¹¹¹In-1033-rituximab (filles squares) antibody conjugates expressed as percentage ±std.dev. The data are corrected for radioactivity decay and background.

FIG. 3 shows comparison of whole blood clearance of radioactivity in rats, injected with ¹¹¹In-1033-trastuzumab (filled triangles) or ¹¹¹In-1033-rituximab (filles squares) antibody conjugates expressed as % of activity at start ±std.dev. The data are corrected for radioactivity decay.

FIG. 4 shows biodistribution of ¹¹¹In-1033-trastuzumab in rats, expressed as % of injected dose per gram tissue ±std.dev. The results are corrected for radiochemical decay.

FIG. 5 shows biodistribution of ¹¹¹In-1033-rituximab in rats, expressed as % of injected dose per gram tissue ±std.dev. The results are corrected for radiochemical decay.

### DESCRIPTION OF PREFERRED EMBODIMENTS

### Definitions

When used in this context “naked antibody” means an antibody, antibody fragments, “Single-chain Fv” anti-body fragments or “diabodies”, which does not carry any agents or structures attached to the immunoglobulin structure in order to enhance the effect of antibody, hence, the effect on tumours cells of the naked anti-bodies need to rely on the intrinsic effect of the anti-body itself.

The term “monoclonal antibody” as used herein refers to an antibody obtained from a population of substantially homogeneous antibodies, i.e., the individual anti-bodies comprising the population are identical except for possible naturally occurring mutations that may be pre-sent in minor amounts. Monoclonal antibodies are highly specific, being directed against a single antigenic site. Furthermore, in contrast to conventional (polyclonal)-antibody preparations which typically include different antibodies directed against different determinants (epitopes), each monoclonal antibody is advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or made by recombinant DNA methods (see, e.g., U.S. Pat. No. 4,816,567). The monoclonal antibodies may also be isolated from phage anti-body libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the reminder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)).

“Humanized” forms of non-human (e.g. murine) anti-bodies are chimeric immunoglobulins. Immunoglobulin chains or fragments thereof (such as Fv, Fab, Fab′, F(ab′)2 or other antigen-binding subsequences of antibodies) which contain a minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are neither in the recipient antibody nor in the imported CDR or framework sequences. These modifications are made to further refine and optimize antibody performance.

In general, the humanized antibody will comprise substantially all of, or at least one, and typically two, variable domains, in which all or substantially all of the CDR regions correspond to those of a non-human immunoglobulin and all or substantially all of the FR regions are those of a human immunoglobulin sequence. The humanized antibody optimally will also comprise at least a portion of an immunoglobulin constant region (Fc), typically that of a human immunoglobulin. For further details, see Jones et al., Nature, 321:522-525 (1986): Reichmann et al., Nature. 332:323-329 (1988): and Presta, Curr. Op. Struct. Biol., 2:593-596 (1992). The humanized antibody produced by immunizing macaque monkeys with the antigen of interest.

“Antibody fragments” comprise a portion of an intact antibody, generally the antigen-binding or variable region of the intact antibody. Examples of antibody fragments include Fab, Fab′. F(ab′)2. and Fv fragments: diabodies; single-chain antibody molecules; and multispecific antibodies formed from antibody fragments.

“Single-chain Fv” antibody fragments comprise the VH and VL domains of antibody, wherein these domains are present in a single polypeptide chain Generally, the Fv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenbourg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow paring between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “anti Erb antibody” used herein is intended to mean an antibody with the ability of specific binding to the various types of mammalian erb gene products expressed on tumour cells, and with an affinity-binding constant of at least 5×10⁻⁶M⁻¹. The term will include, but is not limited to, antibodies against erb1, erb2, erb3 and erb4.

The term erb or erb antigen(s) in this application refers to the various types of the mammalian erb gene products, and in particular the use of these gene products as targets for anti-tumour antibodies.

The term “variants” of the anti Erb antibody as used herein means any modifications, fragments or derivatives thereof having the same or essentially similar affinity-binding constant when binding to the Erb antigen molecule, i.e. an affinity-binding constant of at least 5×10⁶M⁻¹.

Any of these variants could have been modified by the coupling of various numbers of polyethylene glycol chains in order to optimise the half-life in body fluid and the retention of the antibody or antibody fragments or derivatives, in the tumor tissue. In the most preferred application the antibodies or antibody derivatives should allow for the attachment of a sufficient number of biotin residues to be used for extracorporeal removal through interaction with immobilized avidin, without significantly diminishing the binding properties of the targeting agent.

“Treatment” refers to both therapeutic treatment and prophylactic or preventative measures. Those in need of treatment include those already with the disorder as well as those in which the disorder is to be prevented.

“Mammal” for purposes of treatment refers to any animal classified as a mammal, including humans, domestic and farm animals, and zoo, sports, or pet animals, such as dogs, horses, cats, cows, etc. Preferably, the mammal is human.

A “disorder” is any condition that would benefit from treatment with the anti-Erb antibodies. This includes chronic and acute disorders or diseases including the pathological conditions which predispose the mammal to the disorder in question. Non-limiting examples of disorders to be treated herein include benign and malignant tumors; leukemias and lymphoid malignancies; neuronal, glial, astrocytal, hypothalamic and other glandular, macrophagal, epithelial, stromal and blastocoelic disorders; and inflammatory, angiogenic and immologic disorders.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia. More particular examples of such cancers include squarnous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer.

The term “cytotoxic agent” as used herein refers to a substance that inhibits or prevents the function of cells and/or causes destruction of cells. The term is intended to include radioactive isotopes (e.g. I, Y, Lu), chemotherapeutic agents, and toxins such as, but not limited to, active toxins of bacterial, fungal, plant or animal origin, or fragments thereof. Some radionuclides, like indium-111, are used as diagnostic agents and are as such administered with low activity, but could also be used for therapeutical purposes if given in higher doses and are therefore also referred to as cytotoxic agents herein.

A “chemotherapeutic agent” is a chemical compound useful in the treatment of cancer. Examples of chemotherapeutic agents include Adriamycin, Doxorubicin, 5-Fluoruracil, Cytosine arabinoside (“Ara-C”), Cyclophosphamide, Thioptepa, Busulfan, Cytoxin, Taxol, Methotrexate, Cisplatin, Melphalan, Vinblastine, Bleomycin, Etoposide, Ifosfamide, Mitomycin C, Mitoxantrone, Vincristine, Vinorelbine, Carboplatin, Tenisposide, Duanomysin, Caminomycin, Aminopterin, Dactinomycin, Mitomycins, Esperamicins (see U.S. Pat. No. 4,675,187), Maytansinoids, Melphalan and other related nitrogen mustards.

The term “MitraTag™-1033”, also called for short “1033”, as used herein refers to the compound 3-(13′-thioureabenzyl-DOTA)trioxadiamine-1-(13″-biotin-Asp-OH)trioxadiamine-5-isothiocyanato-aminoisophatalate.

The following embodiments of the invention also serve to explain the details of the invention.

All types of cancer expressing Erb gene products on the surface of tumor cells are applicable to treatment with a medical composition, a kit or a method according to the present invention. In a preferred embodiment the medical composition, the kit, or the method, is applied to breast cancer or ovarian cancer. A most preferred application is breast cancer of the so-called HER-2 type, that is breast cancer which over-expresses HER-2. This type is also known as Erb-B2 or c-erb-2.

The present invention presents new medical and pharmaceutical compositions in the treatment of certain types of breast cancer and ovarian cancer in particular.

Furthermore, with the present invention it is possible to improve the tumour to non-tumour ratio of cytotoxic targeting agents in the treatment of disseminated cancer expressing the protooncogene Erb, in particular breast cancer and ovarial cancer, by reducing the concentration of the cytotoxic medical agent in the blood circulation after administrations of a cytotoxic agent and thereby facilitating a higher dosage and hence a more effective treatment regime without exposing the vital organs to higher toxicity.

In one embodiment, a radiolabelled anti Erb antibody is given in a single dose which is limited to what is regarded as tolerable to the patient without reconstitution of the hematopoietic function, through bone marrow transplantation, or by some other means known in the art. The dose range will be 10-20 MBq/kg body weight of ⁹⁰Y-anti Erb antibody (“low dose”), preferably 11-15 MBq/kg, and the range for ¹¹¹In-anti Erb antibody for targeting localisation will be 50-200 MBq/m² body surface, preferably 100-150 MBq/m² body surface. In this embodiment, extracorporeal clearance of non-bound radiolabelled therapeutic or diagnostic antibody is optional.

In another embodiment, a radiolabelled anti Erb antibody is given in a single dose designated to deliver a high amount of radioactivity to the patient. This “high dose method” has to be combined with means to reconstituting the bone marrow or by reducing the radiation effect on bone marrow, preferably by the use of the MitraDep® system. For ⁹⁰Y-anti Erb antibodies, a “high dose” means a single dose exceeding 20 MBq/kg body weight.

In a preferred embodiment, ¹¹¹In-anti Erb antibodies at a dose of 100-150 MBq/m² body surface is combined with a “high dose” (>20 MBq/kg body weight) of ⁹⁰Y-anti Erb antibody, either given in sequence by a time interval of 6-8 days or given simultaneously.

In one embodiment, a radiolabelled anti Erb antibody is given in a single dose which is limited to what is regarded as tolerable to the patient without reconstitution of the hematopoietic function through bone marrow transplantation, or by some other means. The dose range will be 555-2220 MBq/m² body surface of ¹⁷⁷Lu-anti Erb antibody (“low dose”), preferably 1000-2000 MBq/m². In this embodiment, extracorporeal clearance of non-bound radiolabelled therapeutic or diagnostic antibody is optional.

In another embodiment, a radiolabelled anti Erb antibody is given in a single dose designated to deliver a high amount of radioactivity to the patient. This “high dose method” has to be combined with means known in the art to reconstitute the bone marrow or by reducing the radiation effect on bone marrow, preferably by the use of the MitraDep® system. For ¹⁷⁷Lu-anti Erb antibodies, “high dose” means a single dose exceeding 2220 MBq/m² body surface.

The advantages of ¹⁷⁷Lu compared to ⁹⁰Y are the following:

⁹⁰Y is a pure beta-emitter and can not be imaged by external gamma cameras (immunoscintigraphy) and therefore requires the use of ¹¹¹In for imaging. Conversely, ¹⁷⁷Lu emits gamma radiation in addition to beta particle emission. As a result, ¹⁷⁷Lu can be imaged directly, without need for a combination with ¹¹¹In. Therefore, only one radiopharmaceutical is required for localisation and therapy when ¹⁷⁷Lu is used, which will simplify the treatment regime and lower the cost as well as reduce the irradiation burden on the patient.

⁹⁰Y has a shorter physical half-life (2.67 days) and a longer range (12.0 mm) than ¹⁷⁷Lu. The longer half-life (6.7 days) and shorter range (2.2 mm) of ¹⁷⁷Lu offers benefits by allowing a longer time for the antibody-radionuclide to localise to the tumour and the longer half-life also combines well with the long intracellular half-life. In addition, the shorter range of ¹⁷⁷Lu would cause less bystander radiation (cross-fiering) to tissues adjacent to the tumour tissue at the possible cost of less efficacy in bulkier lesions. The longer range of ⁹⁰Y offers benefits in being better able to radiate bulkier lesions.

Breast cancer is staged into five different groups based on the prognosis. Breast cancer happens when cells in the breast begin to grow out of control and can then invade nearby tissues or spread throughout the body. The tumors that can spread throughout the body or invade nearby tissues are considered cancer and are called malignant tumors. Theoretically, any of the types of tissue in the breast can form a cancer, but usually it comes from either the ducts or the glands.

In order to guide treatment and offer some insight into prognosis, breast cancer is staged into five different groups.

### Stage 0 (Called Carcinoma In Situ)

Lobular carcinoma in situ (LCIS) refers to abnormal cells lining a gland in the breast. This is a risk factor for the future development of cancer, but this is not felt to represent a cancer itself.

Ductal carcinoma in situ (DCIS) refers to abnormal cells lining a duct. Women with DCIS have an increased risk of getting invasive breast cancer in the breast. Treatment options are similar to patients with Stage I breast cancers.

Stage I—early stage breast cancer when the tumor is less than 2 cm across and has not spread beyond the breast.
Stage II—early stage breast cancer where the tumor is either less than 2 cm across and has spread to the lymph nodes under the arm; or the tumor is between 2 and 5 cm (with or without spread to the lymph nodes under the arm); or the tumor is greater than 5 cm and has not spread outside the breast.
Stage III—locally advanced breast cancer where the tumor is greater than 5 cm across and has spread to the lymph nodes under the arm; or the cancer is extensive in the underarm lymph nodes; or the cancer has spread to lymph nodes near the breastbone or to other tissues near the breast.
Stage IV—metastatic breast cancer where the cancer has spread outside the breast to other organs in the body.

Although patients representing all five groups could be eligible to treatment according to present invention, in a most preferred embodiment the malignancy represents Stage III and IV.

In the present invention an immunotargeting agent (immunoconjugate) is an agent which carries a cytotoxic moiety that, contrary to common cytotoxic medical agents, binds specifically and with high affinity to tumor cells expressing the protooncogene Erb, and which could be administered to a human being. In a preferred application the immunotargeting agents are antibodies, which could be of different isotypes and could originate from any species. Preferred antibodies are humanised monoclonal antibodies. Furthermore, of particular interest are those, which in addition to the above-described properties bind the erb receptor with an affinity of at least about 50 nM, more preferably at least about 10 nM.

Of particular interest are derivatives of monoclonal antibodies. The latter include fragments such as the Fab, Fab′, F(ab′)2, F(ab″) and Fv fragments and the like. They also include genetically engineered hybrids or chemically synthesized peptides based on the specificity of the antigen binding region of one or several target specific monoclonal antibodies, e.g. chimeric or humanized antibodies, single chain antibodies etc. The biomolecule binding moiety, which is an IgG reactive moiety, is bound or conjugated to the anti Erb antibody, either covalently or non-covalently with an affinity-binding constant of at least 5×10⁸ M⁻¹.

In order to enhance the effect or to introduce diagnostic properties, tumour specific monoclonal antibodies are used as a carrier (immunoconjugates) of various cytotoxic agents, such as, but not limited to, radionuclides, chemotherapeutical agents, synthetic or natural occurring toxins, immunosuppressive or immunostimulating agents, radiosensitizers, enhancers for X-ray or MRI or ultrasound, non-radioactive elements, which can be converted to radioactive elements by means of external irradiation after that the anti Erb antibody carrying said element has been accumulated to specific cells or tissues, or photoactive compounds or compounds used in photo imaging or photodynamic therapy, or any other molecule having the same or a similar effect, directly or indirectly, on cancer cells or cancer tissues, and enzymes used in pro-drug protocols. The cytotoxic agent is preferably a radionuclide, such as a gamma-emitter e.g. iodine-131 or metal ion conjugate, where the metal is selected from a beta-particle emitter, such as yttrium, lutetium or rhenium. U.S. Pat. No. 4,472,509, Gansow et al., discloses the use of diethylenetriaminepentaacetic acid (DTPA) chelating agents for the binding of radio metals to monoclonal antibodies. The patent is particularly directed to a purification technique for the removal of non-bonded and adventitiously bonded (nonchelated) metal from radiopharmaceuticals but is illustrative of art recognized protocols for preparation of radionuclide labelled antibodies.

According to such general procedures, an antibody specifically reactive with the target tissue associated antigen is reacted with a quantity of a selected bifunctional chelating agent having protein binding and metal binding functionalities to produce a chelator/antibody conjugate. In conjugating the antibodies with the chelators, an excess of chelating agent is reacted with the antibodies, the specific ratio being dependent upon the nature of the reagents and the desired number of chelating agents per antibody. It is a requirement that the radionuclides be bound by chelation (for metals) or covalent bonds in such a manner that they do not become separated from the biotinylated/radiolabelling compound under the conditions that the biomolecule conjugate is used (e.g. in patients).

When the cytotoxic agent is a radionuclide, particularly metallic radionuclides, it is bound to the trifunctional cross-linking moiety via a cytotoxic agent binding moiety.

Thus, the most stable chelates or covalent bonding arrangements are preferred. Examples of such binding/bonding moieties, i.e. the cytotoxic agent binding moiety, form aryl halides and vinyl halides for radionuclides of halogens; and comprise N₂S₂ and N₃S chelates for Tc and Re radionuclides; amino-carboxy derivatives such as EDTA, triethylenetetraaminehexaacetic acid and DTPA or derivatives thereof, said DTPA derivatives being Me-DTPA, CITC-DTPA and cyclohexyl-DTPA, and cyclic amines, such as NOTA, DOTA, and TETA, and derivatives (Yuangfang and Chuanchu, Pure & Appl. Chem. 63, 427-463, 1991) for In, Y, Pb, Bi, Cu, Sm, and Lu radionuclides.

Beta radiation emitters, which are useful as cytotoxic agents, include radionuclides, such as scandium-46, scandium-47, scandium-48, copper-67, gallium-72, gallium-73, yttrium-90, ruthenium-97, palladium-100, rhodium-101, palladium-109, samarium-153, lutetium-177, rhenium-186, rhenium-188, rhenium-189, gold-198, and radium-212. The most useful gamma emitters are iodine-131 and indium-m114. Other metal ions useful with the invention include alpha radiation emitting materials such as bismuth-212, bismuth-213, and astate-211 as well as positron emitters such as gallium-68 and zirconium-89.

In another embodiment of the invention, radionuclide-labelled targeting agents are useful not only in the treatment of cancer expressing erb antigens, but also for imaging of such cancers. Imaging can be conducted by the use of β-emitting radionuclides utilizing the brems-strahlung or by γ-emitting radionuclides for imaging. In another preferred embodiment ¹⁷⁷Lu, which is both a β and γ emitter, is used as the cytotoxic agent for both treatment and diagnosing of cancer.

In a preferred embodiment on average 2-4 molecules of the part a)-c) of the conjugate, preferably MitraTag™, are linked to each molecule of the anti Erb antibody, and in the most preferred embodiment the average number of such molecules per anti Erb antibody is 2.5-3.5.

At a suitable time after administration, “cytotoxic targeting agents” will be cleared from the blood system by extracorporeal means. To facilitate the extracorporeal depletion an apparatus for extracorporeal circulation of whole blood or plasma will be connected to the patient through tubing lines and blood access device(s). Such an apparatus should provide conduits for transporting the blood to an adsorption device and conduits for returning the processed blood or plasma to the patient. In the case plasma is processed through the adsorption device, a plasma separation device is needed as well as means of mixing the concentrated blood with processed plasma. The later is normally achieved by leading the two components into an air-trap where the mixing occurs.

In the case where whole blood is processed, an ordinary dialysis machine can constitute the base for such an apparatus. Dialysis machines are normally equipped with all the necessary safeguards and monitoring devices to meet patient safety requirements and allow easy handling of the system. Hence, in a preferred embodiment whole blood is processed and a standard dialysis machine is utilised with only minor modifications of the hardware. However, such a machine requires a new program fitted to the new intended purpose.

In addition to the apparatus, special blood line tubings suitable for the intended flow and distance from the patient and the machine are needed. These line tubings could be made of any material compatible with blood or plasma and would include material used in ordinary tubings used in dialysis.

Blood access could be achieved through peripheral vein catheters, or if higher blood flow is needed, through central vein catheters such as, but not limited to, subclavian or femoral catheters.

For affinity adsorbents, the matrix may be of various shapes and chemical compositions. It may for example constitute a column house filled with particulate polymers, the latter of natural origin or artificially made. The particles may be macroporous or their surface may be grafted, the latter in order to enlarge the surface area. The particles may be spherical or granulated and be based on polysaccharides, ceramic material, glass, silica, plastic, or any combination of these or alike material. A combination of these could, for example, be solid particles coated with a suitable polymer of natural origin or artificially made. Artificial membranes may also be used. These may be flat sheet membranes made of cellulose, polyamide, polysulfone, polypropylene or other types of material which are sufficiently inert, biocompatible, non-toxic and to which the receptor could be immobilized either directly or after chemical modification of the membrane surface. Capillary membranes like the hollow fibers made from cellulose, polypropylene or other materials suitable for this type of membranes may also be used. A preferred embodiment is a particulate material based on agarose and suitable for extracorporeal applications.

In one embodiment Molecularly Imprinted Polymers (MIPs) are used. In such a case the conjugate does not contain any affinity ligands. These are normally cross-linked polymers prepared in the presence of a template molecule. The template can either be molecular structures conjugated to the targeting molecule (chelating groups such as DOTA or DTPA derivatives) or particular structures more or less specific of the targeting molecule (e.g. the antibody structure).

In another embodiment the matrix is coated by ligands which exhibit a specific interaction to the agent (e.g. radio active anti Erb antibody) to be removed from the blood circulation. Such ligands can be chosen from a group comprising monoclonal antibodies including fragments or engineered counterparts thereof, aptamers, peptides, oligodeoxynucleosides including fragments thereof, intercalation reagents including dyestof, oligosaccharides and chelating groups interacting with metals bound to the agent to be removed.

In another embodiment an affinity ligand is attached to the anti Erb antibody and the adsorption device contains an immobilized receptor binding specifically to the affinity ligand. Any type of affinity ligand/immobilized receptor combinations such as “antibodies and antigens/-haptens” and “protein and co-factors” could be used in this application, provided that they exhibit a sufficiently high binding affinity and selectively to the tumor markers and that the affinity ligand-receptor interaction is not interfered with by blood or other body fluids or tissues being in contact with the immunotargeting agent and/or the device.

In one of the most preferred applications, the affinity ligand/immobilized receptor combination is biotin or biotin derivatives and biotin binding molecules, and in particular where the affinity ligand is biotin or derivatives thereof and the immobilized receptor is avidin or streptavidin or any other biotin binding molecule. The affinity ligand pairs of biotin/avidin and biotin/streptavidin are often used with biomolecules. The very strong interaction (i.e. K=10¹³-10¹⁵ M⁻¹) of biotin with the proteins avidin and streptavidin (Green, Methods Enzymol. 184, 51-67, 1990; Green, Adv. Prot. Chem. 29, 85-133, 1975) provides a foundation for their use in a large number of applications, both for in vitro and in vivo uses. A further application of the invention is the simultaneous removal of several different biotinylated “anti-cancer agents” through the same extracorporeal procedure.

One embodiment of the conjugate according to the present invention is in part schematically shown below, wherein the anti Erb reactive moiety is trastuzumab.

The structural requirements for this 1033-conjugate include the biotin containing moiety (the affinity ligand), a linker 1 between biotin and the rest of the molecule, a trifunctional cross-linking moiety, a cytotoxic agent binding moiety, and a linker 2 between the cytotoxic agent binding moiety and the rest of the molecule. The structural requirements of the 1033-conjugate can be split into three parts based on functional requirements. Those parts are the biotin containing moiety, the cytotoxic agent binding moiety, and the trifunctional cross-linking moiety. Formula 1 shows a generalized structure of the inventive conjugate (without any cytotoxic agent bound thereto).

Formula I: Generalized structure for the inventive conjugate intended to bind a metallic radionuclide and containing trastuzumab as the anti Erb antibody.

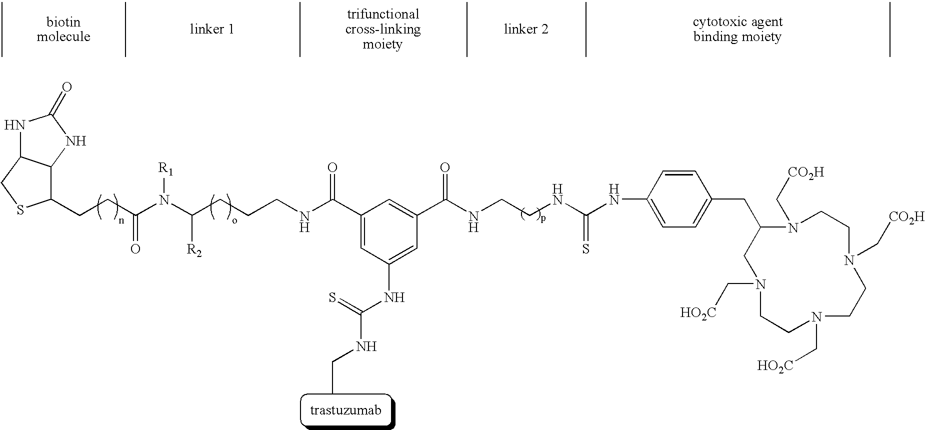

Structural requirements of the biotin containing moiety: There are three aspects of the biotin containing moiety, i.e. the affinity ligand, of the above structure that are important in this context. Those are: (1) blockage of biotinidase cleavage, (2) retention of high biotin binding affinity, and (3) attainment of a reasonable aqueous solubility. To provide those attributes, biotin conjugates must be composed of a biotin molecule and an appropriate linker, which are coupled to a cross-linking moiety.

Biotin conjugates must be prepared by conjugation with the carboxylate on the pentanoic acid side chain (n=3). Conjugation at other locations in the biotin molecule results in complete loss of binding with avidin and streptavidin. This would render the biotin molecule useless for this application. The preferred form of conjugation is formation of an amide bond with the carboxylate group (as depicted in the general formula). Since binding of biotin with avidin and streptavidin takes place in a deep pocket (e.g. 9 Å), shortening (n<3) or lengthening (n>3) of the pentanoic acid side chain results in low binding affinity, which is not desired for this application.

Blocking of the biotinidase activity is achieved by attaching appropriate substituents on the biotinamide amine (i.e. R₁) or on an atom adjacent, i.e. less than three carbon atoms apart, to that amine (i.e. R₂). Biotinidase is an enzyme that cleaves (hydrolyzes) the amide bond of biotin carboxylate conjugates. This enzyme is very important in recycling biotin in animals and man. Metabolism of biotin in (several different) protein carboxylases releases biotin-ε-N-lysine (biocytin), and biotinidase specifically cleaves that amide bond to release free biotin. Biotinidase is also capable of cleaving (non-specifically) other biotinamide bonds. In this application, it is important that biotinidase does not cleave biotin from the conjugates, since otherwise the desired outcome will not be achieved. Thus, the useful biotin conjugate structures incorporate functional groups (R₁ or R₂) that block the enzymatic activity of biotinidase. While it is likely that any structure for R₁ will block biotinidase, its structure is generally limited to a methyl (CH₃) group, as this group completely blocks biotinidase activity. The N-methyl group decreases the binding affinity of biotin with avidin and streptavidin significantly, but it still has use in this application. Larger groups for R₁ (e.g. ethyl, aryl, etc.) are not useful due to the loss of binding affinity. The alternative to having a substituent R₁ is to have a substituent R₂ on the atom (e.g. methylene) adjacent to the biotinamide amine. Much larger and more varied substituents can be used in this position without significant effect on the binding affinity of biotin. Biotinidase is not completely blocked when R₂ is CH₃ or CH₂CH₃, although the rate of cleavage is slowed considerably (i.e. to 25% and 10% respectively). Complete blockage of biotinidase activity is attained when R₂ are —CH₂OH and —CO₂H functionalities. In the case of the —CH₂OH (hydroxymethyl) functionality, such a blocking may be achieved by the introduction of a serinyl group. In the case of the CO₂H (carboxy) functionality, such a blocking may be achieved by the introduction of an α or β aspartyl group. The important consideration is that there is no decrease in binding affinity when these groups are incorporated as R₂. Larger functional groups can also be used as R₂ to block biotinidase activity, but a decrease in binding affinity results. The larger functional groups as R₂ are useful in this application if they do not cause a decrease in binding affinity greater than that obtained when R₁ is CH₃.

The biotin affinity and aqueous solubility of the biotin moiety in the structure of Formula I is affected by the linker moiety used. The length and nature of the linker moiety (linker 1) will be dependent to some degree on the nature of the molecule that it is conjugated with. The linker moiety serves the function of providing a spacer between the biotin moiety and the rest of the conjugate such that the biotin binding is not affected by steric hindrance from the protein (or other conjugated molecule). The length (number of atoms in a linear chain) of linker 1 may vary from o=4-20 for conjugates with small molecules (e.g. steroids) to o>20 for large conjugate molecules (e.g. IgG molecule). The nature of the atoms in linker 1 (linear chain or branch from it) will also vary to increase water solubility. For example, linkers that contain more than 4 methylene units are improved by incorporation of oxygen or sulfur atoms (forming ethers or thioethers) or by having appended ionizable functionalities (e.g. sulfonates, carboxylates, amines or ammonium groups).

Structural requirements of the cytotoxic agent binding moiety: Various radionuclide chelating and bonding agents can be used in the structure of Formula I. In Formula I, a “benzyl-DOTA” moiety is used as an example. Depending on the nature of the cytotoxic agent binding moiety, a linker moiety (linker 2) is required. Some radionuclide chelation and/or bonding moieties have low aqueous solubility, so addition of a linker molecule containing functional groups which improve water solubility is important. In the DOTA chelate, the primary function of the linker moiety is to improve the water solubility of the conjugated molecule. The nature of the atoms in linker 2 (linear chain or branch from it) will vary to increase water solubility. For example, linkers that contain more than 4 methylene units are improved by incorporation of oxygen or sulfur atoms (forming ethers or thioethers) or by having appended ionizable functionalities (e.g. sulfonates, carboxylates, amines or ammonium groups). The length (number of atoms in a linear chain) of linker 2 may also vary (e.g. p=1-20) depending on the nature of hetero atoms incorporated or functional groups appended to the linear chain. Structural requirements of the trifunctional cross-linking moiety: Various trifunctional molecules can be used as the cross-linking moiety. Any molecule that has three functional groups that can be reacted with functional groups on the linkers (linker 1 and 2) and on the protein is a candidate for the trifunctional cross-linking moiety. Aside from the requirement that the trifunctional cross-linking moiety not impart insolubility of e.g. the structure of Formula I in aqueous solutions, the only other structural limitations on the trifunctional cross-linking molecule is that the structure be such that it can be modified in a manner that allows a sequential addition of the biotin containing moiety, and the cytotoxic agent binding moiety, and conjugation with the anti Erb antibody. As an example, a trifunctionalized benzene ring (aminoisophthalic acid) is used in the 1033 structure.

A preferred structure, 1033-trastuzumab, is shown in Formula II below, wherein n=3, o=3, p=3, R₁═H and R₂═COOH (without any cytotoxic agent bound).

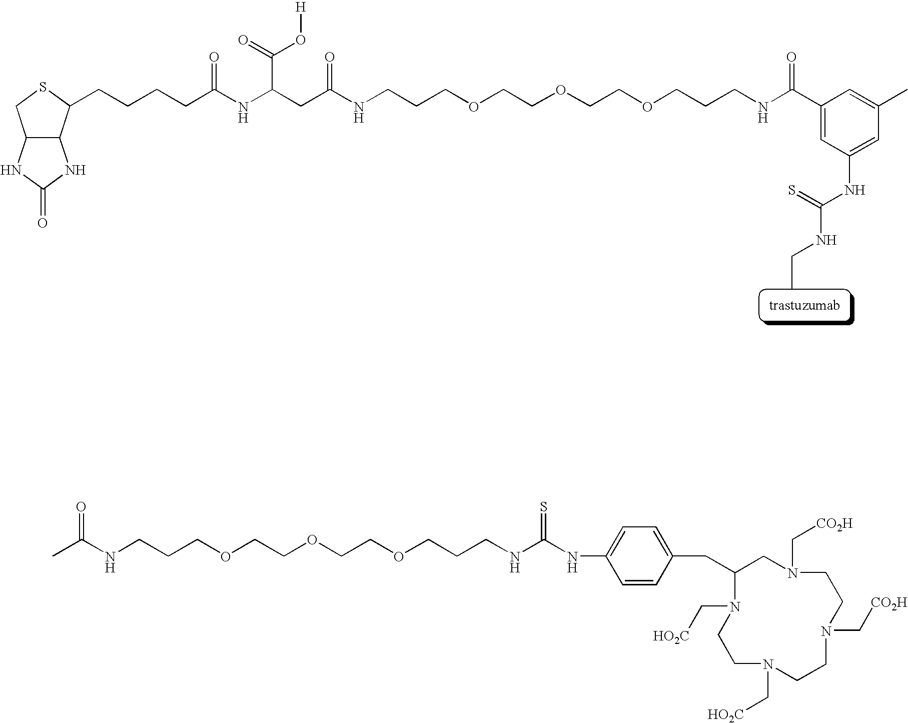

Specific examples of the conjugate according to the present invention are ¹⁷⁷Lu-1033-trastuzumab, i.e. ¹⁷⁷Lu-3-(13′-thioureabenzyl-DOTA) trioxadiamine-1-(13″-biotin-AspOH)trioxadiamine-5-isothiocyanato-aminoisophtalate-trastuzumab; ⁹⁰Y-1033-trastuzumab; ¹¹¹In-1033-trastuzumab; 1033-trastuzumab, wherein thioureabenzyl-DOTA has been replaced with maytansinoid; and 1033-trastuzumab, wherein thioureabenzyl-DOTA has been replaced with doxorubicin.

In another embodiment of the present invention more than one affinity ligand, preferably two, and/or more than one cytotoxic agent, preferably two, are included in the conjugate. In such a case, the cross-linking moiety is more than trifunctional.

### EXAMPLES

The following examples shall not be construed as limiting the invention, but should be regarded as evidence for the applicability of the invention.

### Example 1

### Conjugation and Radiolabelling of Trastuzumab

In this and subsequent examples, Indium-111 has in some instances been used as a substitute for Yttrium-90, because the former is a gamma-emitter and possesses less radiation hazard than Yttrium-90. The monoclonal antibody, trastuzumab, was conjugated with 3-(13′-thioureabenzyl-DOTA)-trioxadiamine-1-(13″-biotin-Asp-OH)-trioxadiamine-5-isothiocyanato-aminoisophtalate (MitraTag™-1033), for short also called “1033” in the following, using the method described by Wilbur D. S et al in Bioconjugate Chem. 13:1079-1092, 2002. A 10 mg quantity of the monoclonal antibody was dialysed against 1 L metal free HEPES with 3 buffer changes over 3 days at 4° C. A solution of MitraTag™-1033 (800 μg) was made in water and was added to the antibody solution. After incubation overnight at room temperature, the antibody-conjugate was dialysed against 1 L metal free 250 mM ammonium acetate buffer pH 5.3 with a minimum of 4 buffer changes over 4 days at 4° C. The average number of MitraTag™-1033 per monoclonal antibody was determined to 2.2 by the HABA method. The demetalated conjugated antibody was stored at 4-8° C. until used in radiolabelling experiments.

Two mg (400 μl) 1033-antibody in 250 mM ammonium acetate (pH 5.3) was mixed with 30 μl of the radionuclide to be studied (¹¹¹InCl₃; ⁹⁰YCl₃; ¹⁷⁷LuCl₃) in 40 mM HCl. The labeling was conducted at 45° C. for 15 minutes. 43 μl of DTPA was added to stop the reaction. The quality of the radio conjugate was determined by TLC and HPLC.

### Example 2

### Binding of the 1033-Conjugated Trastuzumab to an Avidin Adsorbent

The fraction of ¹¹¹In-labelled 1033-trastuzumab radio conjugate binding to the avidin adsorbent utilised in the MitraDep® device was analysed utilising microcolumns. About 97% of the radioactivity in the radiolabelled 1033-conjugate sample was bound to the microcolumn with the avidin adsorbent.

### Example 3

### Analyses of the Affinity of the Binding to the Target Antigen

The influence of the conjugation process on the binding affinity (strength) of trastuzumab to the target antigen was studied utilizing a competitive inhibition assay. Briefly, increasing amounts of trastuzumab were mixed with a constant amount of ¹¹¹In-labelled 1033-trastuzumab. The mixtures were added to fixed SK-BR3 cells in 96 plate wells. After incubation for 2 hours at room temperature, the wells were washed, and the radioactivity bound to the cells was measured in an automatic NaI(Tl) scintillation well counter.

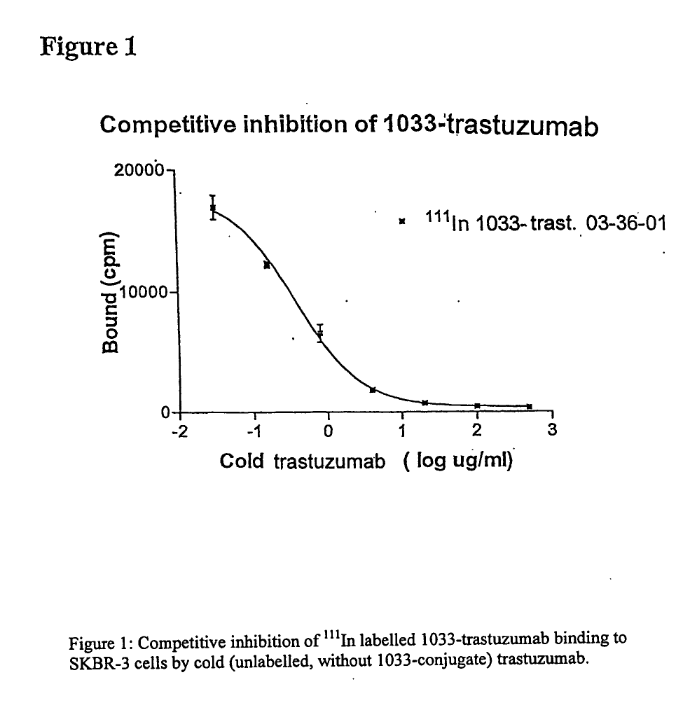

The amount of bound radioactivity was plotted against the concentration of trastuzumab (FIG. 1), and the concentration required for 50% inhibition (IC₅₀) was calculated. The IC₅₀ is a measure of the relative affinity (avidity) of the tested antibody; a decrease of affinity is seen as an increased IC₅₀ concentration. To be a significant change in affinity it is often stated that the difference in IC₅₀ should be at least 10-fold.

1 μg/ml (6.7 nM) of ¹¹¹In-1033-trastuzumab is inhibited by 0.03-500 μg/ml cold non-conjugated trastuzumab. The IC₅₀ was determined to 0.4 μg/ml (2.5 nM). From IC₅₀, the dissociation constant was calculated to 0.3 nM. According to information published by the manufacturer of trastuzumab the dissociation constant is 0.1 nM.

A slight decrease in affinity was seen for the 1033-trastuzumab conjugate. It has been shown in clinical studies that a 10-fold difference in affinity does not result in any significant difference in tumour uptake. Therefore, it was concluded that conjugation of trastuzumab with up to 2.2 conjugates per antibody would not diminish the binding properties of the antibody in vivo.

### Example 4

### Pharmacokinetics of MitraTag™-1033 Conjugate of Trastuzumab

The pharmacokinetics and biodistribution data of ¹¹¹In-1033-trastuzumab is compared to the data obtained with ¹¹¹In-1033-rituximab as clinical data is available for this radio conjugate. Both antibodies are humanized human monoclonal IgG1 antibodies.

Fifteen (15) rats of the Spraque Dawley strain were injected intravenously with approximately 100 μg/rat of 1033-antibody conjugate labelled with 3-4 MBq ¹¹¹In.

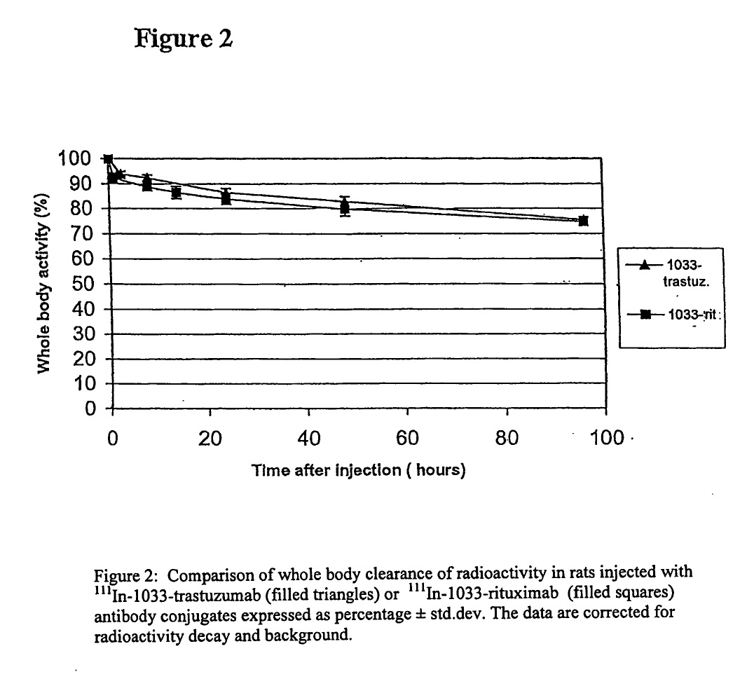

Whole body (WB) imaging was performed using a scintillation camera (General Electric 400T, GE, Milwaukee, Wis., USA) equipped with a medium-energy collimator. Images were stored and analysed with Nuclear MAC 2.7 software. From the images, the total numbers of counts in the entire body were obtained. After radioactivity decay correction and background subtraction, the counts were used for the calculation of activity retention (%) in the body. See FIG. 2.

When whole body retention of ¹¹¹In-1033-trastuzumab was compared to that of ¹¹¹In-1033-rituximab, no significant difference was seen.

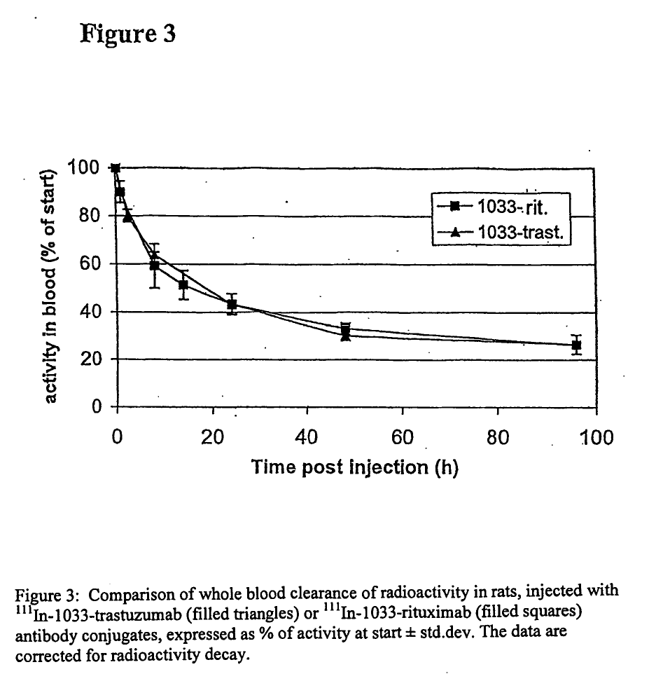

To define pharmacokinetics of ¹¹¹In-1033-trastuzumab and compare it with ¹¹¹In-1033-rituximab, about 0.2 ml blood was obtained from the periorbital venous plexa on the following occasions: 10 min, 2.5, 8, 24, 48 and 96 hours after injection. The radioactivity was measured in an automatic NaI(Tl) scintillation well counter and expressed in percent of injected activity per gram blood (%/g) corrected for ¹¹¹In decay (FIG. 3). When blood clearance of ¹¹¹In-1033-trastuzumab was compared to that of ¹¹¹In-1033-rituximab, no significant difference was seen.

### Example 5

### Biodistribution of Conjugates to Organs and Tissues

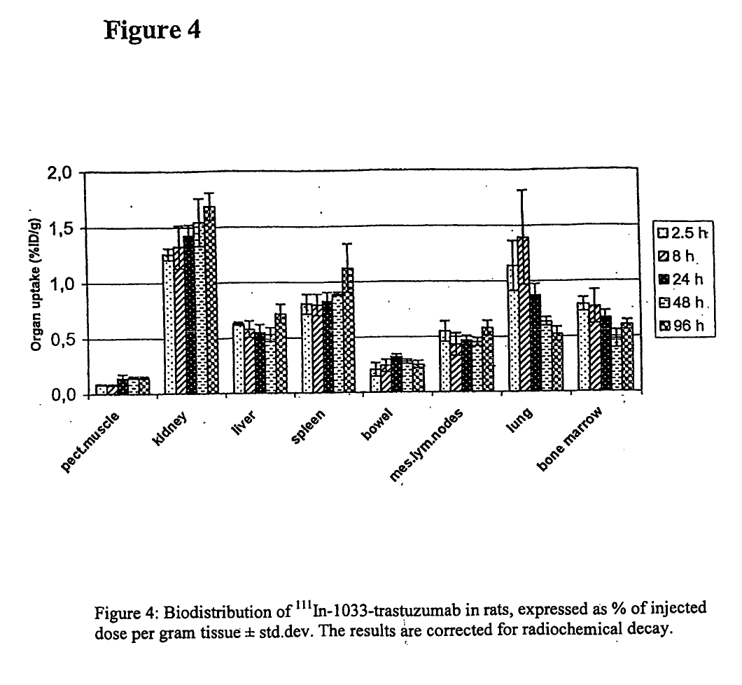

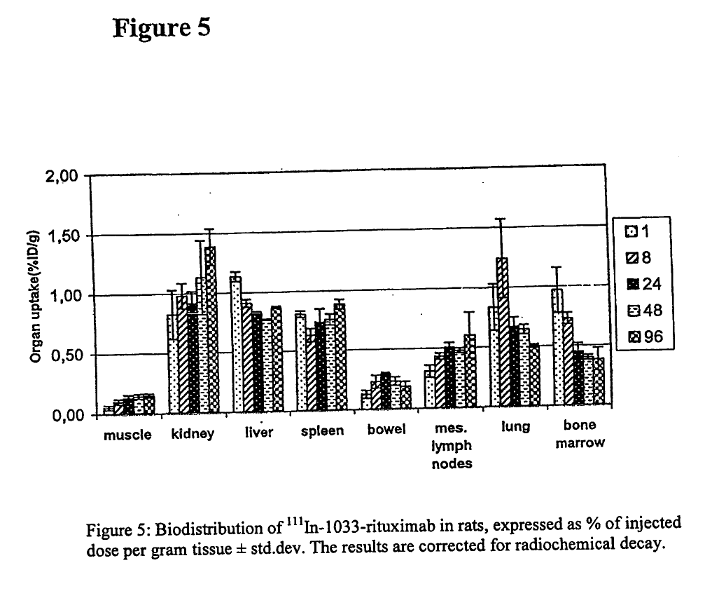

In dissections, performed 2.5, 8, 24, 48, and 96 hours after injection, organs and tissues of interest were removed, weighed and measured for radioactivity content. The radioactivity was measured in an automatic NaI(Tl) scintillation well counter, and the counts were corrected for decay. The distribution to various organs was compared to that of ¹¹¹In-1033-rituximab. The distribution of the injected activity is shown in FIG. 4 (¹¹¹In-1033-trastuzumab) and FIG. 5 (¹¹¹In-1033-rituximab).

A higher uptake in the kidneys and lungs, and a lower in the lungs were seen for ¹¹¹In-1033-trastuzumab compared to ¹¹¹In-1033-rituximab. The higher uptake in the lungs for ¹¹¹In-1033-trastuzumab is mainly observed shortly after injection, ending up at about the same level after 48 hours.

### Example 6

### Treatment Regime with ⁹⁰Y/¹¹¹In-Trastuzumab in Breast Cancer Expressing HER-2 According to a Preferred Embodiment of the Invention

On day 0 all patients will receive 1-4 mg/body weight of trastuzumab immediately followed by a therapeutic dose of ⁹⁰Y-1033-trastuzumab (>10 MBq/kg body weight) Patients may, optionally, be administered a dose of 100-150 MBq/m² body surface (1.1-3.9 mCi/m² body surface) ¹¹¹In-1033-trastuzumab, which will be used for imaging and for dosimetry.
At one occasion during day 1-3, patients are treated with MitraDep®, allowing at least 3 blood volumes to pass the MitraDep® device.
Optionally, and as a safety measure, prior to administration of ⁹⁰Y-1033-trastuzumab, bone marrow may be harvested to allow for bone marrow rescue if required.
Optionally, the treatment can be repeated with ⁹⁰Y-1033-trastuzumab 2-6 times a year and in a most preferred embodiment 2-4 times a year, provided that no dose limiting toxicity has occurred and that the patient has recovered from previous treatment with respect to radiation toxicity.
Optionally, the patient is receiving therapeutic or sub-therapeutic doses of trastuzumab (Herceptin) before or after receiving ⁹⁰Y-1033-trastuzumab and/or trastuzumab (Herceptin) is given in connection with the administration of ⁹⁰Y-1033-trastuzumab.

### Example 7

### Treatment Regime with ¹⁷⁷Lu-Trastuzumab in Breast Cancer Expressing HER-2 According to Another Preferred Embodiment of the invention

At day −7 to day −1 all patients will once receive 6-8 mg/kg body weight of trastuzumab.
On day 0 patients receive a therapeutic dose of ¹⁷⁷Lu-1033-trastuzumab (>555 MBq/m² body surface). Patients may, optionally, be investigated by immunoscintography for imaging and for dosimetry.
At one occasion during day 1-4, patients are treated with MitraDep®, allowing at least 3 blood volumes to pass the MitraDep® device.
Optionally, and as a safety measure, prior to administration of ¹⁷⁷Lu-1033-trastuzumab, bone marrow may be harvested to allow for bone marrow rescure if required.
Optionally, the treatment can be repeated with ¹⁷⁷Lu-1033-trastuzumab 2-6 times a year and in a most preferred embodiment 2-4 times a year provided that no dose limiting toxicity has occurred and that the patient has recovered from previous treatment with respect to radiation toxicity.
Optionally, the patient is receiving therapeutic or sub-therapeutic doses of trastuzumab (Herceptin) before, or after, receiving ¹⁷⁷Lu-1033-trastuzumab and/or trastuzumab (Herceptin) is given in direct connection with the administration of ¹⁷⁷Lu-1033-trastuzumab.

1. A conjugate comprising
a) a trifunctional cross-linking moiety, to which is coupled
b) an affinity ligand via a linker 1,
c) a cytotoxic agent, optionally via a linker 2, and
d) an anti Erb antibody or variants thereof having the ability to bind to Erb antigens expressed on mammalian tumour surfaces with an affinity-binding constant of at least 5×10⁶ M⁻¹, wherein the affinity ligand is biotin, or a biotin derivative having essentially the same binding function to avidin or streptavidin as biotin, wherein stability towards enzymatic cleavage of the biotinamide bond has been introduced in linker 1.

2. The conjugate according to claim 1, wherein the anti Erb antibody or variants thereof are directed to Erb 1, Erb 2, Erb 3, and/or Erb 4 antigens expressed on mammalian tumour surfaces.

3. (canceled)

4. The conjugate according to claim 1, wherein the anti Erb antibody is coupled to the trifunctional cross-linking moiety via a linker 3, and wherein the bond formed between linker 3 and the anti Erb antibody is either covalent or non-covalent with a binding affinity constant of at least 5×10⁸ M⁻¹.

5. The conjugate according to claim 1, wherein the cytotoxic agent is a radionuclide, chemotherapeutical agents, a synthetic or naturally occurring toxin, immunosuppressive or immuno stimulating agents, radiosensitizers, enhancers for X-ray or MRI or ultrasound, non-radioactive elements, which can be converted to radioactive elements by means of external irradiation after the anti Erb antibody carrying said element has been accumulated to specific cells or tissues, or photoactive compounds or compounds used in photo imaging or photodynamic therapy, or any other molecule having the same or a similar effect, directly or indirectly, on cancer cells or cancer tissues.

6. (canceled)

7. The conjugate according to claim 1, wherein when the cytotoxic agent is a radionuclide and is bound to the trifunctional cross-linking moiety via a cytotoxic agent binding moiety.

8. The conjugate according to claim 7, wherein the cytotoxic agent binding moiety form aryl halides and vinyl halides for radionuclides of halogens, and comprises N₂S₂ and N₃S chelates for Tc and Re radionuclides, amino-carboxy derivatives, preferably EDTA, triethylenetetraaminehexaacetic acid, and DTPA or derivatives thereof, wherein the DTPA derivatives are Me-DTPA, CITC-DTPA, and cyclohexyl-DTPA, and cyclic amines, preferably NOTA, DOTA and TETA, and derivatives thereof, for In, Y, Pb, Bi, Cu, Sm and Lu radionuclides, or any other radionuclide capable of forming a complex with said chelates.

9. The conjugate according to claim 7, where in the cytotoxic agent binding moiety comprises DOTA and the cytotoxic agent is ⁹⁰Y for therapeutic application or ¹¹¹In for diagnostic application.

10. The conjugate according to claim 6, wherein the cytotoxic agent binding moiety comprises DOTA and the cytotoxic agent is ¹⁷⁷Lu for both diagnostic and therapeutic application.

11. (canceled)

12. The conjugate according to claim 1, wherein the affinity ligand is a moiety which binds specifically to avidin, streptavidin or any other derivatives, mutants or fragments of avidin or streptavidin having essentially the same binding function to this affinity ligand.

13. The conjugate according to claim 1, wherein the biotin derivative is chosen from the group consisting of norbiotin, homobiotin, oxybiotin, iminobiotin, destibiotin, diaminobiotin, biotin sulfoxide, and biotin sulfone, or derivatives thereof having essentially the same binding function, preferably with an affinity-binding constant of at least 10⁹ M⁻¹.

14. The conjugate according to claim 1, wherein the trifunctional cross-linking moiety is chosen from the group consisting of triaminobenzene, tricarboxybenzene, dicarboxyanyline and diamino benzoic acid.

15-17. (canceled)

18. The conjugate according to claim 1, wherein linker 2 provides a spacer length of 1-25 atoms, preferably a length of 6-18 atoms.

19-20. (canceled)

21. The conjugate according to claim 1, wherein linker 3 provides a spacer of a length of 1-25 atoms, preferably a length of 6-18 atoms, or groups of atoms.

22-25. (canceled)

26. The conjugate according to any one of the preceding claims, wherein it is 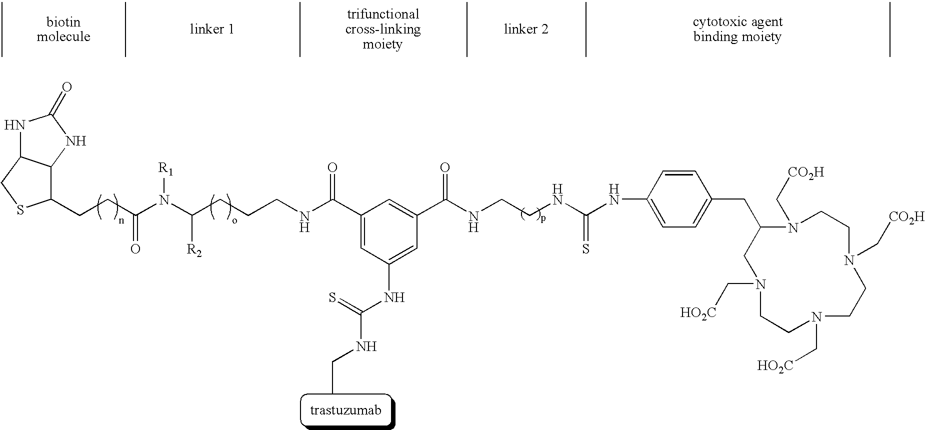
wherein n is 2-4, o is 1-6, p is 1-6, R₁ is H, and R₂ is —COOH, and wherein n preferably is 3, o preferably is 3, and p preferably is 3, bound to a cytotoxic agent via the cytotoxic agent binding moiety.

27. The conjugate according to claim 1, wherein it is ¹⁷⁷Lu-1033-trastuzumab, i.e. ¹⁷⁷Lu-3-(13′-thioureabenzyl-DOTA)trioxadiamine-1-(13″-biotin-Asp-OH) trioxadiamine-5-isothiocyanato-aminoisophtalate-trastuzumab; ⁹⁰Y-1033-trastuzumab; ¹¹¹In-1033-trastuzumab; 1033-trastuzumab, wherein thioureabenzyl-DOTA has been replaced with maytansinoid; and 1033-trastuzumab, wherein thioureabenzyl-DOTA has been replaced with doxorubicin.

28. A medical composition, wherein it comprises the conjugate according to claim 1 together with a pharmaceutically acceptable excipient.

29. (canceled)

30. A kit for extracorporeal removal of or at least reduction of the concentration of a non-tissue bound medical composition as defined in claim 28, comprising a conjugate according to claim 1, in the plasma or whole blood of a mammalian host, wherein said medical composition has previously been introduced in the body of said mammalian host and kept therein a certain time in order to be concentrated to the specific tissues or cells by being attached thereto, said kit comprising
a) said medical composition, and
b) an extracorporeal device comprising an immobilized receptor onto which the affinity ligand of the conjugate adheres.

31. The kit according to claim 30, wherein it comprises antibodies and antigens/haptens or protein and cofactors as affinity ligand/immobilized receptor combinations, preferably biotin or biotin derivatives as affinity ligands and avidin or streptavidin as the immobilized receptor.

32. The kit according to claim 30, wherein the affinity ligand is absent in the conjugate of the medical composition, and the immobilized receptor is molecularly imprinted polymers interacting with the conjugate.

33. A method for the treatment of cancer expressing Erb gene products on the surface of its tumour cells in a mammalian host, wherein a medical composition according to claim 28 is administered to the mammal in need thereof.

34. The method according to claim 33, wherein said cancer is breast or ovarian cancer.

35-37. (canceled)

38. A method for diagnosing cancer expressing Erb gene products on the surface of its tumour cells in a mammalian host, wherein a medical composition according to claim 28 is administered to the mammalian host.

39. The method according to claim 38, wherein said cancer is breast or ovarian cancer.

40-41. (canceled)

42. A method for treatment and diagnosing of cancer expressing Erb gene products on the surface of its tumour cells in a mammalian host, wherein a medical composition according to claim 28 and 29 containing ¹¹¹In in a dose of 50-200 MBq/m₂ body surface, preferably 100-150 MBq/m₂ body surface, and a medical composition according to claim 28 containing ⁹⁰Y as a cytotoxic agent in a dose of 10-20 MBq/kg body weight, preferably 11-15 MBq/kg body weight, are administered to the mammalian host.

43. A method for treatment and diagnosing of cancer expressing Erb gene products on the surface of its tumour cells in a mammalian host, wherein a medical composition according to claim 28 containing ¹¹¹In in a dose of 100-150 MBq/m₂ body surface, and a medical composition according to claim 28 containing ⁹⁰Y as the cytotoxic agent in a dose of more than >20 MBq/kg body weight, are administered to the mammalian host, either in sequence in said order by a time interval of 6-8 days or simultaneously.

44. A method for treatment and diagnosing of cancer expressing Erb gene products on the surface of its tumour cells in a mammalian host, wherein a medical composition according to claim 28 containing ¹⁷⁷Lu as the cytotoxic agent in a single dose of 555-2220 MBq/m₂ body surface, preferably 1000-2000 MBq/m₂ body surface, is administered to the mammalian host.

45. A method for treatment and diagnosing of cancer expressing Erb gene products on the surface of its tumour cells in a mammalian host, wherein a medical composition according to claim 28 containing ¹⁷⁷Lu as the cytotoxic agent in a single dose of more than 2220 MBq/m₂ body surface is administered to the mammalian host together with means to reconstitute the bone marrow or by reduction of the radiation effect on the bone marrow.
